(19) United States
(12) Patent Application Publication
(10) Pub. No.: US 2026/0351398 A1
(43) Pub. Date: Nov. 5, 2026

(54) REMOTE MEDICAL DEVICE AUTHENTICATION

(71) Applicant: CareFusion 303, Inc., San Diego, CA (US)

(72) Inventor: Kevin O’BRIEN, Thurles (IE)

(21) Appl. No.: 19/197,478

(22) Filed: May 2, 2025

(51) Int. Cl. G16H 40/40; A61M 5/172; H04L 9/08; H04L 9/32

(52) U.S. Cl. CPC G16H 40/40; A61M 5/172; H04L 9/085; H04L 9/3236; H04L 9/3297; A61M 2205/3584; A61M 2205/502; A61M 2205/60

(57) ABSTRACT

Certain aspects of the disclosure provide systems and methods for verification of updates to remote medical devices. For example, methods may include obtaining an update to an operational parameter of a medical device, outputting a first code, and obtaining a second code based on the first code and the update to the operational parameter. Based on verification of the second code, the operational parameter may be updated.

### BACKGROUND

### Field

Aspects of the present disclosure relate to remote medical device authorization.

### Introduction

Various medical devices are used for various purposes, such as to administer therapeutics to patients. One example medical device is an infusion pump configured to infuse therapeutic fluid into a patient. Generally, an infusion pump is programmed to pump fluid at a specific flow rate and up to a specific volume. The infusion pump then operates according to one or more operational parameters to administer the fluid. The operational parameters are configured such that a correct dose of fluid is administered, e.g., volume of fluid, and during the correct time frame, e.g., flow rate, to ensure proper dosing of the fluid for patient safety.

During operation of the infusion pump, a clinician may enter the operational parameters, for example, using physical inputs at the infusion pump itself. In other cases, a control message may be sent to the infusion pump, for example, from another device with the operational parameters, such as a clinician using a communication device to send the control message, e.g., through a wired or wireless connection.

After initial set up, the infusion pump may be locked or control access restricted to prevent tampering or accidental adjustment to the operational parameters. However, sometimes one or more of the operational parameters may need to be updated, for example, based on a change in patient condition. A clinician or other authorized personnel may need to unlock the infusion pump to input the updates to the operational parameters. This may include using an access badge scanned by the infusion pump, using credentials (e.g., username/password), an authorization code, or similar mechanisms to control operations of the infusion pump.

Such security messages restrict access to the infusion pump to prevent unauthorized adjustments, such as tampering or accidental adjustment. For example, erroneous control messages or malicious control messages may be prevented from being implemented where such messages are not sent from an authorized device or authorized individual. On the physical device, inputs may be locked out, e.g., control buttons restricted, such that new input is only received after authentication of a user. However, such measures may unduly burden authorized access by preventing or burdening authorized users from properly adjusting the infusion pump.

### SUMMARY

Certain aspects provide a method comprising obtaining input indicative of an update to at least one operational parameter of a medical device; outputting a first code; obtaining a second code that is based at least on the first code and the update to the at least one operational parameter; and based on verification of the second code with respect to the first code and the update to the at least one operational parameter, updating the at least one operational parameter based on the input.

Certain aspects provide a method comprising obtaining a first code, wherein the first code is associated with an update to at least one operational parameter of a first medical device; generating a second code based at least on the first code and the update to the at least one operational parameter; and outputting the second code.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1 depicts an example verification system for verification of updates to remote medical devices.

FIG. 2 depicts an example workflow for verification of updates to remote medical devices.

FIG. 3A depicts an example workflow for verification of a first code associated with an update to a remote medical device.

FIG. 3B depicts an example workflow for generation of a second code associated with the verification of the first code.

FIG. 3C depicts an example workflow for verification of the second code.

FIG. 4A depicts example data associated with the first code.

FIG. 4B depicts example data associated with generation of the second code.

FIG. 4C depicts example data associated with verification of the second code.

FIG. 5A depicts an example user interface associated with updating a medical device.

FIG. 5B depicts another example user interface associated with the first code.

FIG. 5C depicts another example user interface associated with the second code.

FIG. 5D depicts another example user interface associated with the update to the medical device.

FIG. 5E depicts another example user interface associated with the update to the medical device.

FIG. 6 depicts an example infusion system.

FIG. 7 depicts an example syringe infusion system.

FIG. 8 depicts an example method for updating an operational parameter of a medical device.

FIG. 9 depicts an example method for verification of an update to an operational parameter of a medical device.

FIG. 10 depicts an example computing device with which aspects of the present disclosure can be performed.

FIG. 11 depicts another example computing device with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for remote medical device authorization. Certain aspects are discussed herein with respect to an infusion system as an example medical device, and associated operational parameters of an infusion system are discussed. However, the techniques discussed herein are also applicable to other types of medical devices, and their associated operational parameters.

As described, infusion pumps, and other medical devices, may have lock or restriction measures to prevent improper access, e.g., malicious, or erroneous control of the device. However, such measures may also restrict or burden proper access.

For example, in remote care scenarios, such as home care or remote site care, an authorized clinician may not always be physically present to unlock the device to adjust the operational parameters (e.g., using physical input at the device or a wired connection). When a clinician desires to adjust the operational parameters of a device in remote care, the clinician could instruct the patient or a caregiver to make the adjustments themselves. The clinician would then need to provide credentials or an access code to the patient or caregiver to unlock the device, and then the patient or caregiver may adjust the operational parameters. There is a risk that once the patient or caregiver as the credentials or access code, the patient or caregiver could make changes other than those instructed by the clinician. For example, the clinician may desire to change the flow rate of the infusion. The patient uses the access code given by the clinician to adjust the flow rate. However, now that the patient has the access code, the patient could adjust the volume of the infusion, or make other improper adjustments.

Another way to overcome physical distance is to have a clinician (such as a health care provider or pharmacist) program a second device and ship or deliver the second device to the patient with the new operational parameters. However, due to shipping and delivery constraints, there may be a delay in implementation of the new operational parameters. Further, there would be waste due to: the patient having two devices, the need for extra devices to be available to ensure that devices are available to be sent to patients, and the cost associated with shipping and delivery of additional devices. Also, by having two devices, there is a risk of mix-up by using the wrong device with the old operational parameters instead of the new device.

In some cases, a clinician may travel to the patient to physically input the new operational parameters on the device, without needing to share access codes or send an additional device to the patient. However, this requires the clinician to spend time traveling to the patient's remote location, for a short visit to make the updates. Some remote locations may be impractical or nearly impossible for clinical travel.

Further, there is a desire to verify and record updates to operational parameters. For the above mentioned methods, which may include manual recordation of a communication to a patient, recordation of shipment of additional devices, nurse visit records, or the like to record updates. As for verification of the updates, patients, caregivers, or clinicians may video call or send photos of the device with the updates implemented to record and verify the updates were implemented. For example, some organizations may desire to have peer or clinician verification of the update by reviewing a video call of the device updates or a photograph of the device settings. The verification of the updates may then also be manually recorded. These methods rely on trust and may be prone to human errors.

An alternative method may include using on-device connectivity to allow a remote clinician to send a control message through a wireless connection (e.g., over the internet), to adjust operations of the device. This overcomes challenges and risks with traveling clinicians, sending additional devices, and giving patients and caregivers access codes or credentials. However, connected devices may be subject to cyber-security challenges because a device configured to receive remote control messages may receive erroneous or malicious remote messages. Additionally, some remote locations may not have reliable wireless connectivity, such as limited internet connectivity or inconsistent internet access, whereby a remote control message may not always be receivable by the device.

Aspects of the present disclosure provide a solution to these technical limitations through a dual code system which enables reciprocal adjustment and verification of operational parameters on a medical device and a clinician device. For example, a user inputs one or more operational parameters on the medical device and receives a first code, which may also be referred to herein as a challenge code. The user may communicate the challenge code to a clinician, even using unsecured, conventional methods such as telephone calls, text messages, satellite calls, etc. In some cases, a clinician may input the one or more operational parameters and the challenge code at a clinician device. The clinician device, may then, generate a second code, which may also be referred to herein as a response code based on the operational parameters and the challenge code. The clinician may communicate the response code to the user, which is inputted to the medical device. The response code is verified and the one or more operational parameters are implemented.

Beneficially, using the dual code system, the user and clinician may communicate remotely, even with unsecured or conventional communication channels such as telephone calls, text messages, satellite, or internet communications, which are readily available and common.

Further, the dual codes may be relatively short such that the codes themselves are easily communicated over these communication channels. The codes themselves, however, may be meaningless without access to the medical device or the clinician device because each device may be equipped with a (e.g., hash) function configured to verify and generate codes using the function, and in some cases, a shared secret key. In certain aspects, without the function and, in some cases, the shared secret key, either code cannot readily be generated, ensuring access is restricted to those with a properly generated code.

Additionally, the codes may be time-limited whereby even a correct code is limited in time to update operational parameters. Thereby, beneficially, a user with an old code cannot later update operational parameters. Also, each code may be limited by the operational parameters, ensuring that only the correct operational parameters are implemented at the medical device.

Moreover, the clinician device may be connected to a hospital information system to facilitate documentation and recordation of the clinician authorization and verification of the operational parameters, as well as indication of the implementation of the update to the medical device. Thereby, even remote updates to the medical device may be recorded in a patient's medical record.

### Example Remote Medical Device Verification System

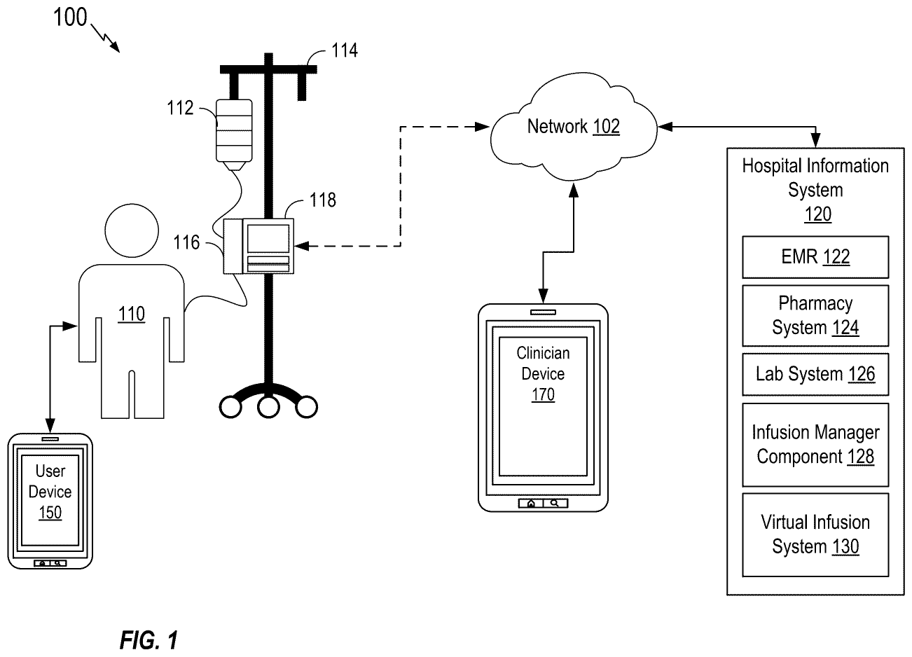

FIG. 1 depicts an example system 100 for verifying updates to remote medical devices, such as the infusion system 114, coupled to a patient 110.

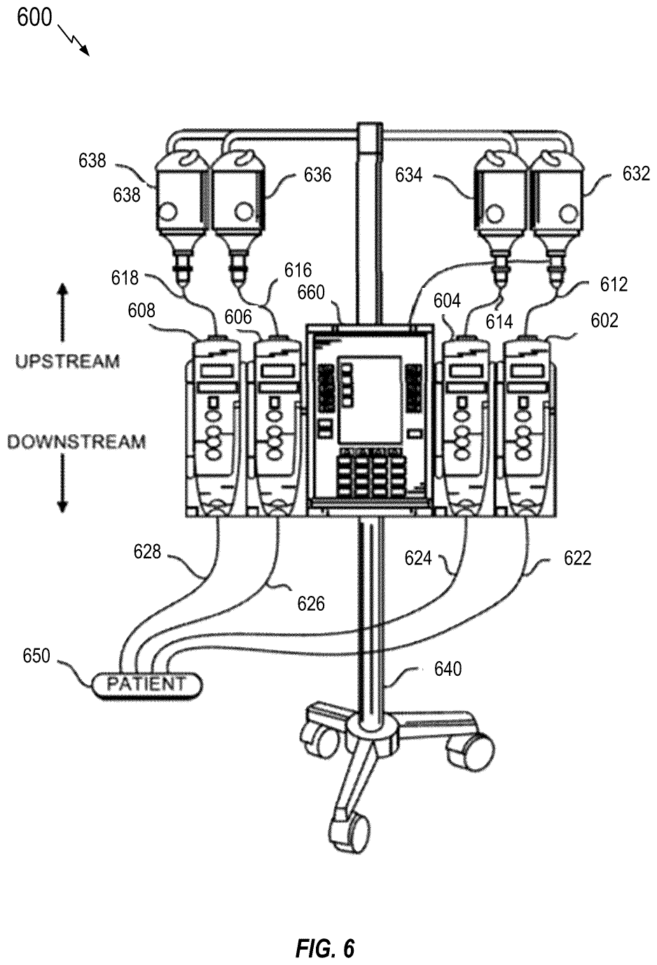

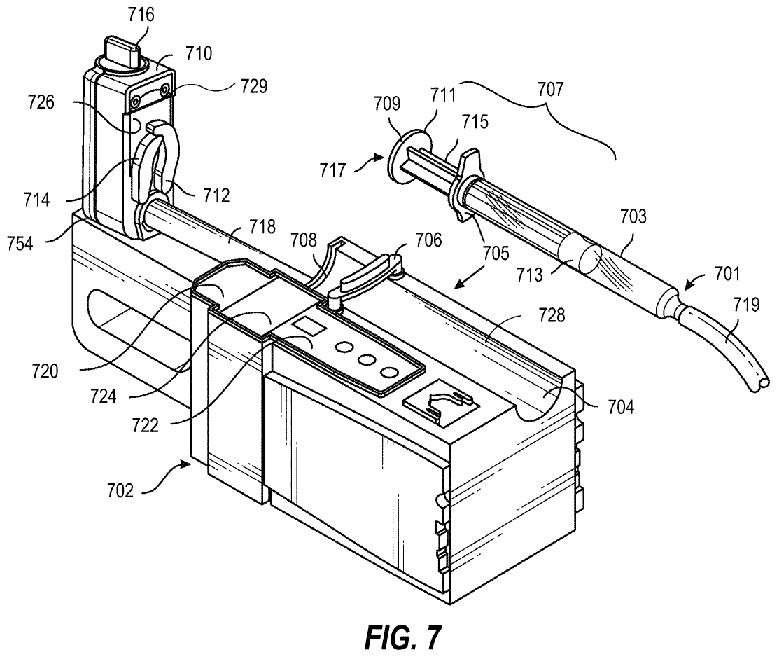

Infusion system 114 is an electromechanical device for infusing therapeutic fluids into the patient 110 in a controlled and precise manner. Exemplary infusion systems may include a large volume pump or a syringe pump, such as depicted in FIG. 6 or FIG. 7. Specifically, in this example, infusion system 114 includes one infusion pump 116. Infusion system 114 comprises an infusion system controller 118 configured to control operations of infusion system 114. Infusion system controller 118 may be an example of a pump control unit (PCU) or pump control device (PCD).

In general operation, therapeutic fluid is loaded into the infusion pump 116, for example, in a syringe, bottle, bag, or other fluid container 112, and the infusion system 114 is configured to pump, or infuse, the therapeutic fluid according to operational parameters of the infusion pump 116, including, for example, flow rate, volume, and/or time to administer a specific dose of treatment. As described in detail with respect to FIG. 6 or FIG. 7, below, the infusion system controller 118 and/or the infusion pump 116 may include one or more buttons, controls, displays, keys, or the like to control operations of the infusion system 114. In some examples, a user may utilize the infusion system controller 118 to enter operational parameters or otherwise control operation of the infusion pump 116. Further, the infusion system controller 118 may display drug entries from the drug library. In certain aspects, each of the associated sets of drug delivery parameters includes information selected from a group of parameters including drug concentration, drug delivery rate, drug dose, and/or bolus size. The electronically loaded drug library may contain a list of available mode options specifying the units available for expressing drug delivery information, and the drug infusion pump may offer the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers.

In some aspects, the infusion system controller 118 may contain a memory comprising a drug library or libraries, an event log or logs, and pump configuration settings, such as, but not limited to, profiles to be used in particular care areas such as ICU, PED, etc. The memory may be electronically loadable memory such as non-volatile memory (e.g., EEPROM). Drug libraries stored on infusion systems (which illustratively contain such information as the drug names, ranges of delivery parameter values such as proper concentrations, dosage units, and dose limits) may be used to perform drug calculation-based infusions in a clinical setting. A drug library stored within the infusion system may include clinical order settings such as limits set by the clinical institution for each drug of the library (also referred to as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward (e.g., a care area) of the medical institution in which the drug is being used (for example neonatal care unit (NICU), the intensive care unit (ICU), etc.), and depending on other factors. An alarm may be provided if the clinician sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

Infusion system 114 is further configured to transmit and receive data, such as data related to the infusion including infusion parameters, infusion data, operational data, or the like. In some aspects, infusion system 114 may be “remote,” that is, geographically located separately from a medical care center, and/or not connected to network 102.

A hospital information system (HIS) 120 is an information system associated with the medical care center of the patient's clinician. The HIS 120 is configured with an electronic medical recordation (EMR) system 122, a pharmacy system 124, a laboratory system 126, or the like, for the medical center, to facilitate communications such as transmitting and receiving laboratory testing data, prescription data, documentation of a patient's medical history, diagnosis, treatments, laboratory data, medical device status, or the like. HIS 120 may include, or be configured to interface with, other hospital systems such as an admissions system, a billing system, a central supply system, unit station systems, or a medical decision support system.

In some aspects, the pharmacy system 124 includes a formulary and/or pharmacy information system. Pharmacy information systems may enable a safer physician medication order process. A pharmacy website (e.g., provided by the server) may provide the physician with a list of available drugs from which the physician may select. The pharmacy website may contain a drug library having the list of available drugs but may also contain and present to the physician the drug names associated with recommended dosages and dose limits that have been established or adopted by the medical center. In such a case where the physician need only select items from the computer screen rather than having to manually type in drug names and drug administration numbers (such as infusion rates, times, etc.) associated with administration of the medication, a more accurate medication process may result.

If a clinical order is for administration of a particular medication regimen, the order will be transmitted to the medical center's pharmacy information system 124. The pharmacy reviews the order, and once the order has been prepared, the order may be transmitted to a nurse station for matching with the appropriate patient. A formulary generally refers to an approved list of drugs for use (e.g., available to order for a patient) within a medical facility. Within a formulary, there may be indication for use information and/or concentrations and drug ranges approved for the medical center. A formulary may be used to define one or more medical device drug libraries, which may then be provided to infusion pumps associated with HIS 120. Inside the library, there is medication information such as drug names, concentration, diluent volume, strength, minimum or maximum infusion parameters for a drug, and other parameters. The establishment of these parameters, along with parameters for off-formulary orders, via the network 102, is useful for maintaining consistency across the medical center environment and ensuring an order is intelligible and executed according to expectations by other devices within the HIS 120 (e.g., an infusion system 114).

Where the clinical order is for a therapeutic fluid to be infused, e.g., with infusion system 114, the order may be called an infusion order, and prescribe a dose of the therapeutic fluid, and a time period for administration. In some aspects, an infusion order may further include operational parameters to infuse the dose in the time period prescribed.

In this example, the HIS 120 includes an infusion manager component 128 and virtual infusion system 130. Although depicted here as part of HIS 120, in some cases, the infusion manager component 128 and the virtual infusion system 130 may be separate from the HIS 120, in data communication with the HIS 120, e.g., via network 102. In some cases, the infusion manager component 128 and the virtual infusion system 130 may form part of the clinician device 170, such as a mobile device, a nurse station monitoring device, a computing device, or the like. The clinician device 170 is configured to facilitate communications with patient 110 or a caregiver of patient 110 through user device 150. For example, the clinician device 170 and the user device 150 may communicate through various communication channels, such as telephone communications, text messages, satellite communications, internet communications, or other communication channels.

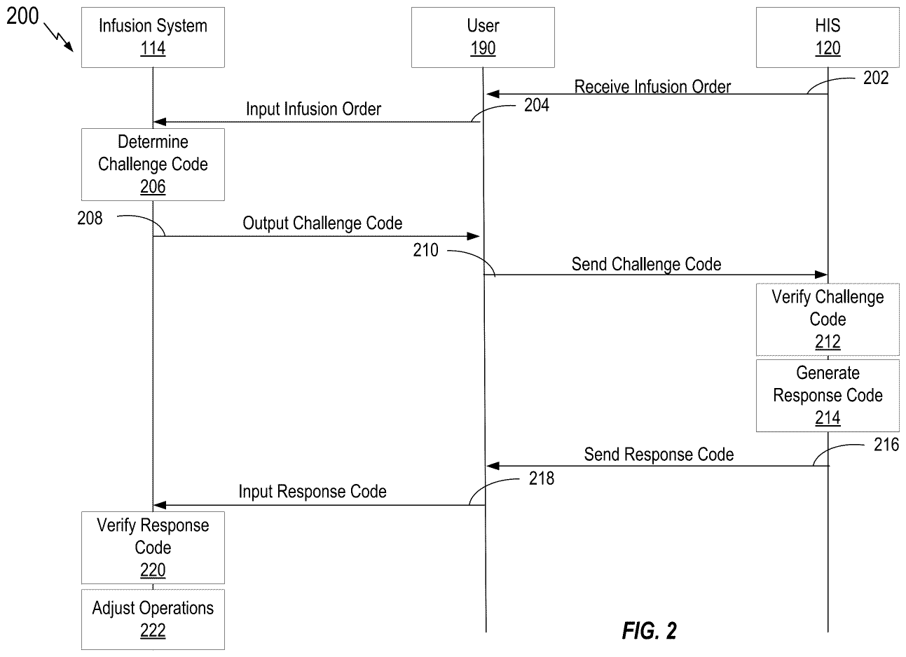

The infusion manager component 128 is configured to facilitate implementation of updates on the remote medical device, infusion system 114. In certain aspects, and as described further with respect to FIG. 2, the infusion manager component 128 is configured to receive and verify a challenge code, via clinician device 170, generated by the infusion system 114, and generate a response code based on verification of the challenge code.

In some aspects, the infusion manager component 128 is configured to utilize virtual infusion system 130 to verify an infusion order for the infusion system 114. In some aspects, virtual infusion system 130 may comprise or utilize a drug library or libraries, an event log or logs, and pump configuration settings, such as, but not limited to, profiles to be used in particular care areas such as ICU, PED, etc. Drug libraries may be used to perform drug calculation-based infusions in a clinical setting. A drug library may include clinical order settings such as limits set by the clinical institution for each drug of the library (also referred to as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward (e.g., a care area) of the medical center in which the drug is being used (for example neonatal care unit (NICU), the intensive care unit (ICU), etc.), and depending on other factors. An alarm may be provided if the clinician sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the virtual infusion system may record each such limit event for later analysis where the attempted setting is higher than the maximum or lower than the minimum dosage. In some aspects, one or more drug libraries associated with virtual infusion system 130 may be readily updated and/or revised as part of HIS 120.

Network 102 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

### Example Workflow for Verification of Updates at Remote Medical Devices

FIG. 2 depicts an example workflow 200 for verifying updates at a remote medical device, for example, at the infusion system 114 in FIG. 1.

Initially, at step 202, a user 190, for example, a user of the user device 150 in FIG. 1, receives an infusion order to update one or more operational parameters of the infusion system 114. In some aspects, the user 190 is the patient 110, or a caregiver of the patient 110. The infusion order may be received though a variety of communication channels, such as telephone communications, text messages, satellite communications, internet communications, or other communication channels. For example, the user device 150 may receive a text message indicating a flow rate of the infusion system 114 is to be updated to 100 mL/hr. As another example, the user 190 may receive a telephone call via the user device 150 from a clinician to update the volume of the infusion system 114 to be 300 mL.

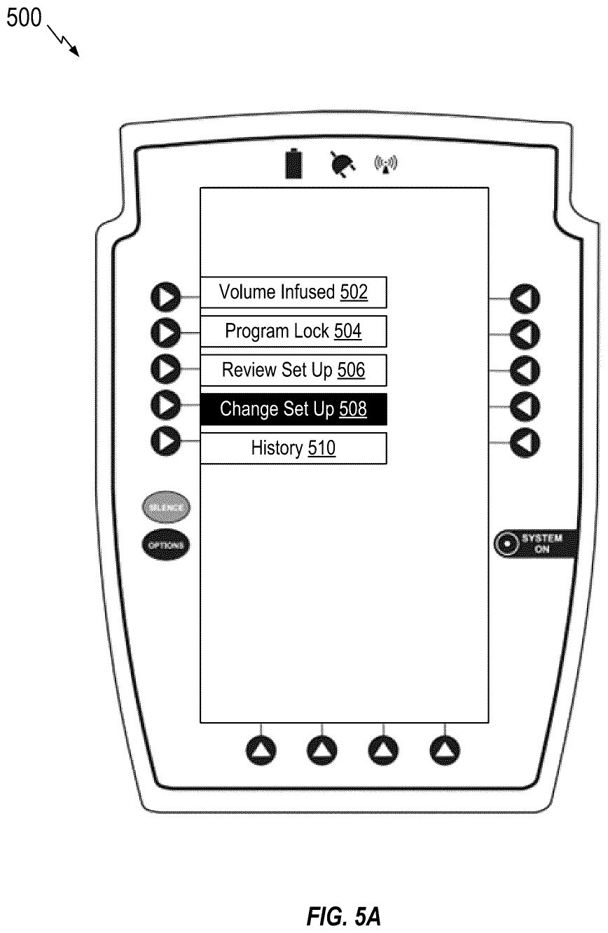

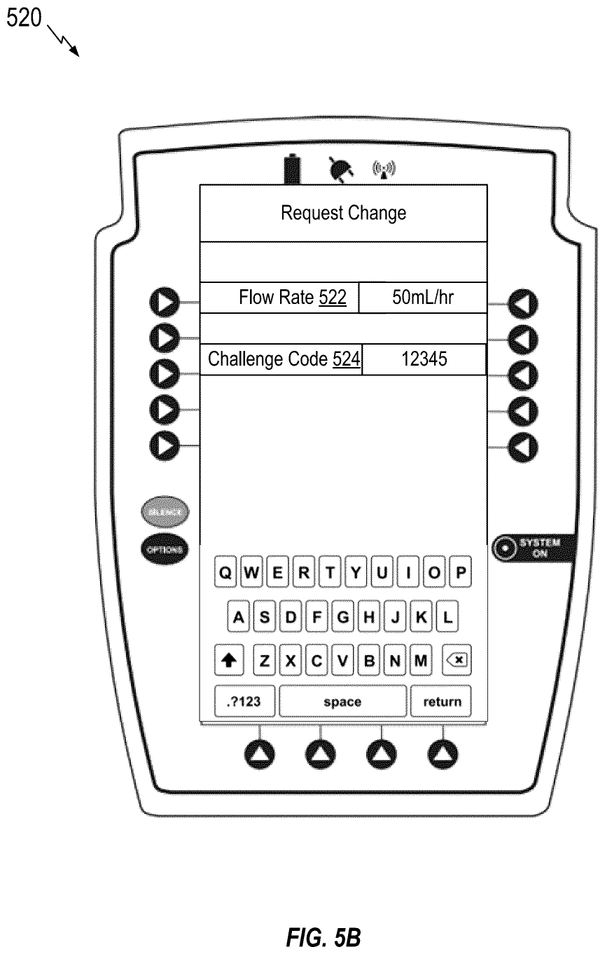

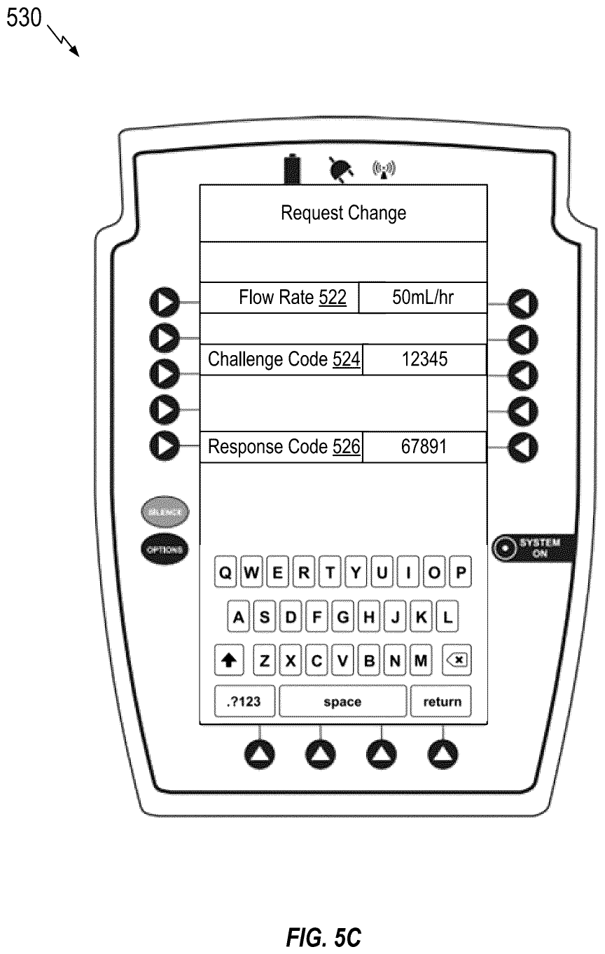

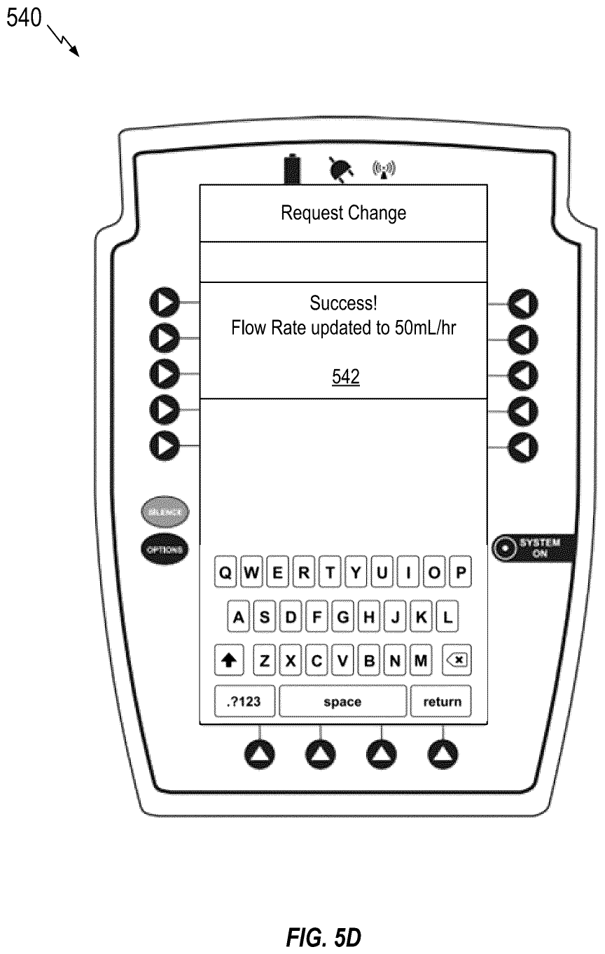

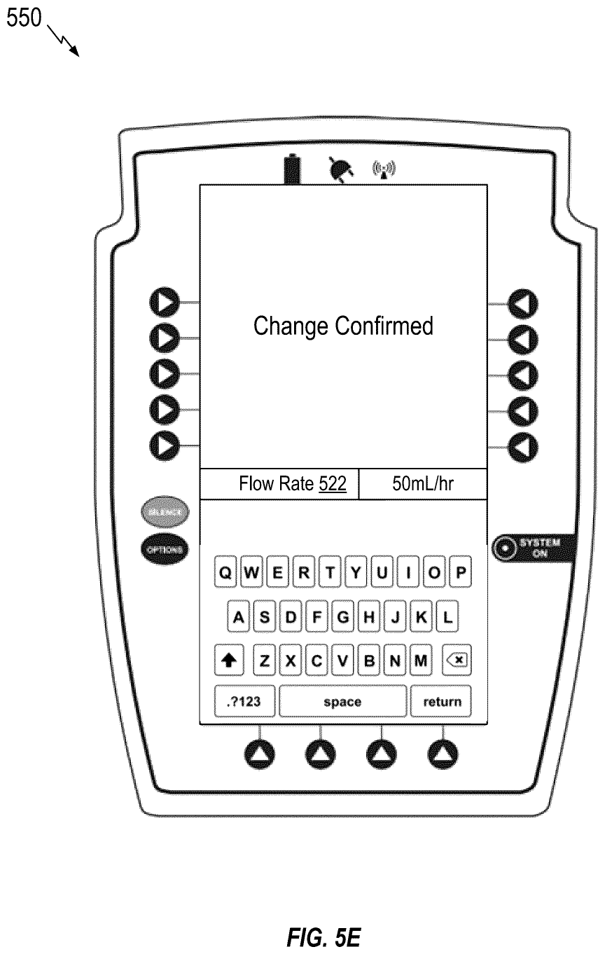

At step 204, the user inputs the infusion order to the infusion system 114, for example, though a user interface of the infusion system 114. FIG. 5A depicts an example user interface 500 of an infusion system, specifically in this example, on an infusion system controller 118. The user interface 500 includes several control elements including a volume infused control element 502, a program lock control element 504, a review set up control element 506, a change set up control element 508, and a history control element 510. Additional, fewer, or other control elements are possible. A user may select change set up control element 508 to input a change or update to the operational parameters of the infusion system 114.

Returning to workflow 200, at step 206, the infusion system 114 determines a challenge code. In some aspects, the challenge code may be a random numeric or text string generated by the infusion system 114. In some aspects, the challenge code may be a sequential numeric or text string generated by the infusion system 114. For example, a sequential numeric string may represent a number of previous update requests, a number of previous successful updates (e.g., updates implemented at infusion system 114), a number of challenge codes generated, or the like.

At step 208, the infusion system 114 outputs the challenge code, for example, by displaying the challenge code on the user interface of the infusion system 114. In some aspects, the infusion system 114 may output a message indicating the challenge code, for example, to a nearby user device, such as user device 150.

FIG. 5B depicts an example user interface 520 of the infusion system controller 118 through which the user inputs an update to the flow rate element 522, here, 50 mL/hr. After entry of the update to the flow rate, a challenge code element 524 may populate, in this example as challenge code 123456.

At step 210, the user 190 may use the user device 150 to send the challenge code to a clinician at the medical center. In some aspects, the clinician receiving the challenge code may be the same clinician that sent the infusion order. In some aspects, the clinician receiving the challenge code may be a different clinician from the one who sent the infusion order, for example, to enable a peer verification of the infusion order.

The challenge code may be sent through a variety of communication channels, such as telephone communications, text messages, satellite communications, internet communications, or other communication channels. For example, the user device 150 may send a message, such as a text message, an email message, satellite message, etc. indicating the challenge code to a clinician. As another example, the user may communicate the challenge code via a telephone or internet call. In some examples, the user and the clinician may remain on a telephone call while the user inputs the update to the operational parameter and communicates the challenge code back to the clinician.

Beneficially, the challenge code may be a short string, for example, less than 10 characters whereby it can be easily communicated over a variety of communication channels.

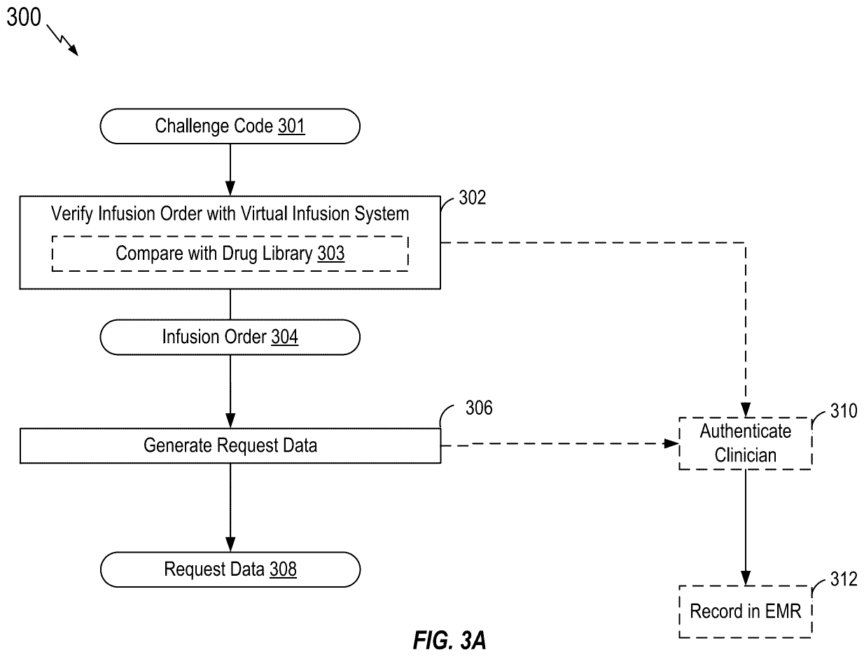

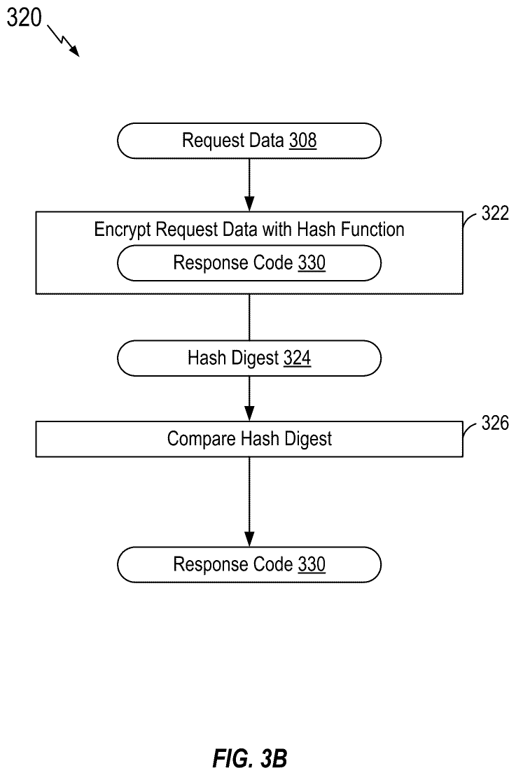

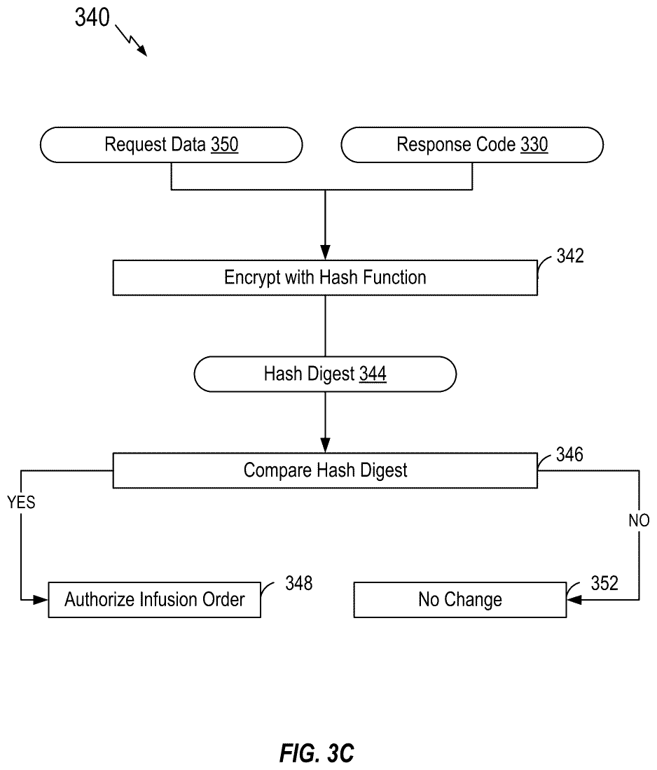

At step 212, the challenge code is verified. As described further with respect to FIG. 3A, the challenge code may be verified at the HIS 120, for example, the infusion manager component 128. In some aspects, verification of the challenge code may include verification of the infusion order, for example, the update to one or more operational parameters.

At step 214, the HIS 120 generates a response code. As described further with respect to FIG. 3B, the response code is generated at the HIS 120, for example, the infusion manager component 128. The response code may be generated based on the challenge code and the infusion order. Then, the response code may be outputted, for example, in some aspects, the response code may be outputted at a clinician device, for example, on a display of a clinician device.

The response code may be a numeric string, for example, a short numeric string, for example, less than 10 characters whereby it can be easily communicated over a variety of communication channels.

At step 216, the response code is sent to the user for input to the infusion system 114. The response code may be sent through a variety of communication channels, such as telephone communications, text messages, satellite communications, internet communications, or other communication channels. For example, the user device 150 may receive a message, such as a text message, an email message, satellite message, etc. indicating the response code to the user. As another example, the clinician may communicate the response code via a telephone or internet call. In some examples, the user and the clinician may remain on a telephone call while the clinician verifies the challenge code and generates the response code for communication back to the user.

At step 218, the user inputs the response code to the infusion system 114, for example, though a user interface of the infusion system 114. FIG. 5C depicts an example user interface 530 of the infusion system controller 118 through which the user inputs the response code, here 67890, at a response control element 526.

Returning to workflow 200, at step 220, the infusion system 114 verifies the response code. As described further with respect to FIG. 3C, the response code is verified by the infusion system 114. The infusion system 114 may verify the response code based on the challenge code generated by the infusion system 114 and the infusion order.

At step 222, the infusion system 114 updates the operational parameter of the infusion system 114, for example, by implementing the operational parameter or saving the updated operational parameter. Then, the infusion system 114 may adjust operations of the infusion system 114 according to the infusion order. FIG. 5D depicts an example user interface 540 of the infusion system controller 118 displaying a message 542 indicating the flow rate update was successfully implemented. FIG. 5E depicts an example user interface 550 confirming the change before exiting a change set up mode to change or update to the operational parameters of the infusion system 114.

Note that workflow 200 is just one example, and other flows including fewer, additional, or alternative steps, consistent with this disclosure, are possible.

### Example Workflow for Verifying a Challenge Code

FIG. 3A depicts an example workflow 300 for verifying a challenge code, such as at step 212 in workflow 200, such as with an infusion manager component 128.

Workflow 300 begins with receiving a challenge code 301, for example, by a clinician inputting the challenge code via a clinician device 170. In some aspects, the challenge code 301 may be received directly from a communication received at a clinician device 170. For example, where the clinician and the user are communicating via message, such as a text message, an email message, a chat message, or the like, the challenge code may be received in the message.

At step 302, workflow 300 proceeds with verifying the infusion order, including one or more updated operational parameters, with a virtual infusion system 130. The infusion order is verified to determine the infusion order is within a permissible range, or satisfies thresholds associated with the virtual infusion system 130. The thresholds may comprise safety thresholds, or mechanical thresholds for infusions and infusion systems.

In some aspects, the entire infusion order, e.g., all operational parameters of the infusion order are verified. In some aspects, only a portion of the infusion order, e.g., one or more parameters to be updated, is verified.

In some aspects, the portion of the infusion order to be verified may be based on an indicator of the entire infusion order encoded in the challenge code. In some aspects, the portion of the infusion order to be verified may be based on an indicator of a change to the infusion order (e.g., the one or more updated parameters) encoded in the challenge code. In some aspects, the portion of the infusion order to be verified may be based on an indicator of the current parameter(s) of the infusion device and the updated parameter(s) of the infusion device.

In some aspects, verifying the infusion order with the virtual infusion system 130 includes step 303, comparing the infusion order with a drug library associated with the virtual infusion system 130. As described herein, a drug library may comprise infusion information, such as drug names, ranges of delivery parameter values such as concentrations, dosage units, and dosage limits, such as maximum and minimum dosages. The virtual infusion system 130 may represent infusion system 114, for example, the alarms or limits associated with the virtual infusion system 130 may be the same as the infusion system 114.

In some aspects, one or more drug libraries associated with the virtual infusion system 130 may be more frequently updated compared to a drug library of the infusion system 114. Because the infusion system 114 may be remote, e.g., not connected or less frequently connected to the HIS 120 (e.g., through network 102), the infusion system 114 may be less frequently updated. Thus, the virtual infusion system 130 may utilize a more updated version of a drug library when verifying the infusion order.

Where the infusion order satisfies the limits associated with the drug library of the virtual infusion system 130, the infusion order may be verified. In some aspects, where the infusion order may satisfy a hard limit (e.g., within a maximum hard limit and a minimum hard limit within the drug library) but not a soft limit, the virtual infusion system 130 may alert or alarm and the clinician may override the alert or alarm.

The verified infusion order 304 is used to generate request data 308 at step 306. The request data 308 may comprise the challenge code, the infusion order, and/or other information about the infusion order or system. For example, the request data 308 may include an identifier of the infusion system 114, such as a serial number or unique device identifier. Information associated with infusion order may include the operational parameter(s) to be updated, current operational parameters, an identifier of the fluid of the infusion, or the like.

In some aspects, the request data 308 may further include a time indicator. A time indicator may indicate a timestamp associated with input of the update to the infusion order, or a timeslot associated with a response time for entry of a response code. For example, a timeslot may comprise a period of time from the input of the update to the infusion order to expiration of the update, whereby the correct response code may be entered to implement the update. Beneficially, a timeslot may time-limit the update to the infusion system. In particular, the time-limit may be a short period in which a response code cannot be readily guessed or predicted, preventing unauthorized access to the infusion system because brute force guessing of the response code at the infusion device may be time and resource intensive beyond the time-limit. Further, the time-limit may be a short period in which an old response code cannot be later verified, such as to implement a previous update. Thereby, security of the infusion system in increased.

In some aspects, the timeslot may be based on a criticality or risk of the update to the infusion system. For example, a large update, e.g., a large difference between a current operational parameter and the updated operational parameter may indicate a high risk of the update and may be associated with a short timeslot. As another example, an update to the operational parameter may be indicated as critical or urgent, and may be associated with a short timeslot to ensure the update is implemented quickly. Other updates which may not be deemed critical or a small update may have a large timeslot. In some aspects, a timeslot may be a period of 15 minutes, 30 minutes, 12 hours, 24 hours, or the like.

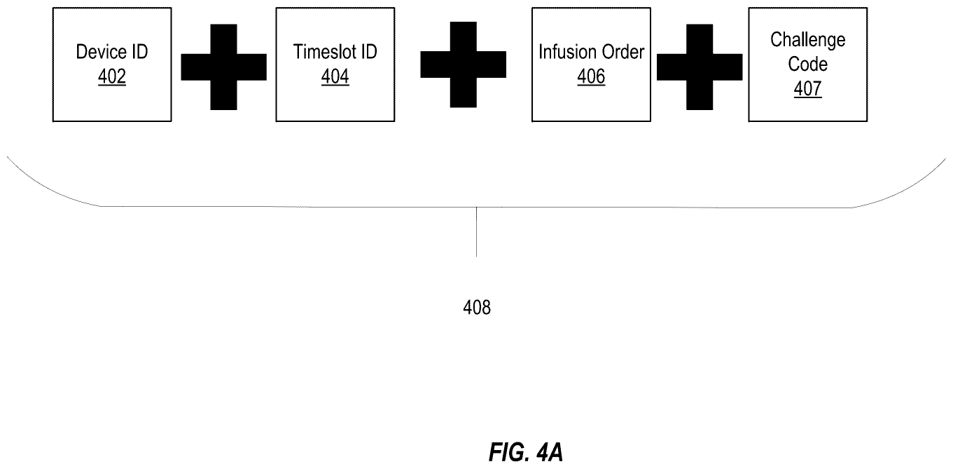

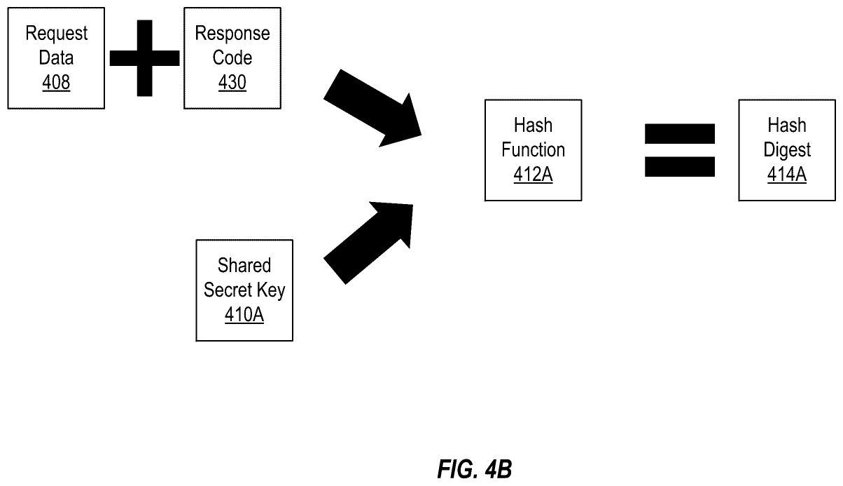

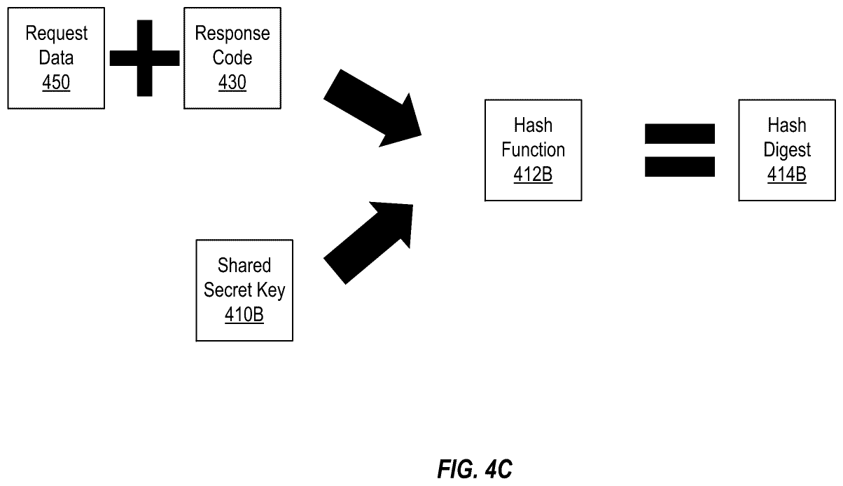

FIG. 4A depicts example request data 408 comprising information indicating the device identifier 402 indicating the device, a timeslot identifier 404 indicating the timeslot for verification of the response code, an infusion order 406 indicating the infusion order information, such as the updated operational parameter, current infusion information, or the like, and the challenge code 407.

In some aspects, workflow 300 continues with step 310 whereby the clinician entering the challenge code is authenticated, for example, by logging into an account, using credentials, etc.

The authenticated clinician may digitally sign a message comprising the infusion order, verification of the challenge code, etc. to indicate the clinician approved the update.

For example, in some aspects, where the clinician overrode an alert or alarm of the virtual infusion system 130, the clinician override may be indicated in a message. The message may also indicate the operational parameter which did not satisfy the soft limit, e.g., triggering the alert or alarm.

At step 312, the message may be recorded in an EMR of the patient 110, for example, by transmitting the message to the EMR system 122.

In some aspects, the challenge code may be recorded in the EMR of the patient 110, for example, as part of the message, or with a data storage associated with the infusion manager component 128. In some aspects, the challenge code may indicate a number of updates of the infusion system 114. For example, where the challenge code is sequential, the challenge code may represent a number of previous update requests, a number of previous successful updates, a number of challenge codes generated, or the like, of the infusion system 114. For example, if an immediately prior challenge code ended in 14, and the present challenge code ends in 14, this may indicate that the prior update was not successfully implemented because the same sequential number is present in the challenge code.

Note that workflow 300 is just one example, and other flows including fewer, additional, or alternative steps, consistent with this disclosure, are possible.

### Example Workflow for Generating a Response Code

FIG. 3B depicts an example workflow 320 for generating a response code, such as at step 214 in workflow 200. In some aspects, the response code is generated by the infusion manager component 128 at the medical center.

Workflow 320 begins at step 322 with encrypting the request data 308 and a response code 330 with a hash function to generate a hash digest 324. The request data 308 and the response code 330 may be encrypted using a shared secret key to generate a hash digest 324. The shared secret key may be shared with the infusion system 114. In some examples, the shared secret key may be specified for the infusion system 114, and a different secret key is shared with different medical devices.

A cryptographic hash function is a mathematical function that maps input data to a fixed-size output, called the hash digest. The hash digest represents the input data. The hash digest is the same size regardless of the size of the input data. Hash functions are one-way such that it is computationally infeasible with conventional (e.g., non-quantum) computing systems to revert the hash digest back to the input data.

For example, a hash function may be used to encrypt the request data 308, the response code 330, and the shared secret key to generate the hash digest 324. FIG. 4B depicts the request data 408 and the response code 430 combined, e.g., the response code 430 appended to the request data 408, and the shared secret key 410A are hashed using the hash function 412A to generate the hash digest 414A.

Additionally, in some aspects, the hash function may be dynamic, for example, by rotating hash functions, using a device-specific hash function, patient-specific hash function, or other hash functions specific to a user, device, care center, etc. to increase security.

At step 326, workflow 320 proceeds with comparing the hash digest 324 with an expected pattern. If the hash digest 324 does match the expected pattern, the response code 330 is outputted. If the hash digest 324 does not match the expected pattern, then the response code 330 is not outputted, and a new response code is generated such that the hash digest 324 will match the expected pattern. In some aspects, the expected pattern may comprise a particular sequence of binary values present in the output (e.g., the hash digest 324).

Note that workflow 320 is just one example, and other flows including fewer, additional, or alternative steps, consistent with this disclosure, are possible.

### Example Workflow for Verifying a Response Code

FIG. 3C depicts an example workflow 340 for verifying a response code, such as at step 220 in workflow 200, at the infusion system 114.

Initially, at step 342, the response code 330 and request data 350 are encrypted with a hash function to generate a hash digest 344. Specifically, the response code 330 is inputted at the infusion system 114 based on a communication from the clinician. The infusion system 114 generates the request data 350 based on the challenge code determined at step 206 and the infusion order information inputted at the infusion system 114 at step 204. Further, the request data 350 may include a time identifier (e.g., a time period) and a device identifier of the infusion system. Beneficially, the request data 350 should be the same as the request data 308 generated by the infusion manager component 128 using the virtual infusion system 130, but is generated at the infusion system 114. Where the request data 350 and the request data 308 are the same, the infusion system 130 has verified the same infusion order as inputted at the infusion system 114.

A time identifier may include an identifier of the time period for which the response code 330 is valid, for example, 10 minutes, 15 minutes, 30 minutes, etc., which the response code is to be entered otherwise the response code may not be verified. The infusion system 114 and the HIS 120 may both be preconfigured with a time period for which the request code is valid. The request code may include an identifier of the time which the request code was generated (e.g., start time), and the time period is added to the start time, whereby if the current time of verification is beyond the time period, the response code is not verified. Beneficially, the time identifier may add an additional security measure because it limits the time interval in which response codes could be brute force entered. Generally, the time interval may be short enough that such brute force attack would be unlikely to guess the correct response code, but long enough to allow the user to receive the response code and enter it into the infusion system.

The request data 350 and the response code 330 may be encrypted using a shared secret key to generate a hash digest 344 at the infusion system 114. The shared secret key may be shared with the infusion manager component 128. The hash function is also shared with the infusion manager component 128, such that when the request data 350, the response code 330, and the shared secret key are hashed, the hash digest is the same at the infusion system 114 as at the infusion manager component 128 at the medical center.

For example, FIG. 4C depicts the request data 450 and the response code 430 combined, e.g., the response code 430 appended to the request data 450, and the shared secret key 410B are hashed using the hash function 412B to generate the hash digest 414B, where the shared secret key 410B is the same secret key as 410A, but located on a memory of the infusion system 114, and the hash function 412B is the same hash function as 412A, but also located on the memory of the infusion system 114. Thus, the hash digest 414B is also the same as the hash digest 414A, but generated at the infusion system 114 instead of the infusion management component 128 at the medical center.

At step 346, workflow 340 proceeds with comparing the hash digest 344 with an expected pattern. In some aspects, the expected pattern may comprise a particular sequence of binary values present in the output (e.g., the hash digest 324).

At step 346, if the hash digest 344 matches the expected pattern, then workflow 340 proceeds to step 348 with authorizing the infusion order.

At step 346, if the hash digest 344 does not match the expected pattern, then workflow 340 proceeds to step 352 with not changing the infusion, e.g., not implementing the infusion order.

Note that workflow 300 is just one example, and other flows including fewer, additional, or alternative steps, consistent with this disclosure, are possible.

### Example Infusion System

FIG. 6 depicts an example infusion system 600, which may implement methods described herein for updating operational parameters. The infusion system 600 may be coupled to a patient 650 to administer a substance to the patient 650 through infusion. The infusion system 600 may include one or more pumps, for example, pump 602, pump 604, pump 606, and pump 608. Although a large volume pump is illustrated, other types of pumps may be implemented, such as a peristaltic pump, a small volume pump, a syringe pump, an anesthesia delivery pump, or a patient-controlled analgesic. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 602, 604, 606, or 608 may comprise a standard infusion pumping unit, patient-controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+mL/hr).

Each of the pumps 602, 604, 606, or 608 may be fluidly connected with an upstream fluid line 612, fluid line 614, fluid line 616, and fluid line 618, respectively. Further, each of pump 602, pump 604, pump 606, and pump 608, may be fluidly connected with a downstream fluid line 622, fluid line 624, fluid line 626, and fluid line 628, respectively. The fluid lines may be any type of fluid conduit, such as tubing, through which fluid can flow.

Each of fluid supply 632, fluid supply 634, fluid supply 636, and fluid supply 638, may be a reservoir, for example, as bottles shown, inverted and suspended above the pumps. Fluid supplies may also take the form of bags, syringes, or other types of containers. The medical device system 600 may be mounted on a roller stand or intravenous pole 640.

Infusion pump system 600 may further comprise check valves, drip chambers, valved ports, connectors, and other devices configured to administer a substance.

Each of the pumps 602, 604, 606, or 608 of infusion pump system 600 may be coupled to an infusion pump controller 660. The infusion pump controller 660 may be a patient care device (PCD) or patient care unit (PCU). The infusion pump controller 660 is configured to control operations of the pumps 602, 604, 606, or 608, for example, to control infusions to patient 650. The infusion pump controller 660 may be further configured to the control of fluid delivery to the patient and the monitoring of the fluid path for occlusion or air-in-line obstructions.

The infusion pump controller 660 may further include a graphical user interface (GUI), for example, an information display such as a liquid crystal display, and/or a touch-screen. The user interface of the infusion pump controller 660 may be used during set up and/or operating procedures to facilitate control of the infusion system 600. For example, entry, editing, and/or display of operational parameters of the pumps 602, 604, 606, or 608. The infusion pump controller 660 may further include one or more softkeys and/or hardkeys to facilitate data entry and commands. In some aspects, a user interface may display the operational parameters of the pumps 602, 604, 606, or 608, for example, the infusion rate at which the pump is operating. The user interface may additionally and/or alternatively, be used to display informational, advisory, alarm, or malfunction messages associated with the pumps 602, 604, 606, or 608.

The infusion pump controller 660 may further be configured to provide power to the infusion system 600 and interface between aspects of the infusion system 600 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the infusion pump controller 660 is configured to provide physical attachment of the infusion system 600 to structures, such as intravenous pole 640 and/or bedrails, and the like.

### Example Syringe Pump

FIG. 7 depicts an example syringe pump 702. Syringe pump 702 includes a graphical user interface (GUI) 724 for displaying operational information, including the selected syringe type and the configured infusion (e.g., in terms of rate of infusion, volume infused, length of time of infusion, etc.). A user may operate syringe pump 702 through one or more buttons 722 in this example. Note that other examples may include other arrangements, such as touch-screen interfaces, or interfaces operable by remote device, such as by an application on a computing device.

Syringe 701 is shown next to syringe pump 702, rather than loaded in the pump, for clarity of illustration. Syringe pump 702 includes cradle 704 in which a barrel 703 of syringe 701 rests when mounted in the syringe pump 702. Sensor 728 detects volume markings along a barrel of syringe 701. Cradle 704 has a clamp 706 to securely hold the barrel 703 in a fixed position in cradle 704 to resist axial and lateral movement. Clamp 706 may pivot between an open position to permit loading or removal of syringe 701 and a closed position over cradle 704. Barrel clamp 706 may measure an outside diameter of the barrel 703 of syringe 701. Barrel flange 705 of syringe 701 resides in a barrel flange groove 708 in syringe pump 702 to immobilize barrel 703 from axial movement during movement of plunger 707 within barrel 703 of syringe 701. Barrel flange groove may measure an outside diameter and a thickness of the barrel flange 705 of syringe 701.

Plunger 707 can include push-button 709 having an inner side 711 and being interconnected with stopper 713 of plunger 707 by piston 715. When mounted in syringe pump 702, push-button 709 can be held by drive head 710 with a plunger retainer comprising a pair of pivotally mounted claws, first retainer claw 712 and second retainer claw 714, shown in the closed position in FIG. 7. The retainer claws 712 and 714 can curve inwardly toward each other to grasp push-button 709 mounted in syringe pump 702.

A rotation knob 716 can be used to control the positions of the first and second retainer claws 712 and 714 to allow removal and insertion of the push-button 709 and to release the split-nut from the driveshaft to permit axial positioning of the drive head 710. Syringes can be provided for use with a syringe pump with different quantities of fluid, and the plunger can be located at different positions in relation to the barrel.

The drive head 710 can allow manual adjustment to accommodate syringes with different beginning plunger positions. A syringe inserted in the cradle 704 can align with the drive head 710 within a particular axial range. The points where the axial center lines of the syringe intersect the driver can change according to the size of the syringe but only in one direction along the drive head 710. A guide device 718 can extend from the drive head 710 to a point within a body of the syringe pump 702.

Syringe pump 702 can include a control panel 720 providing multiple buttons 722 for control of the syringe pump 702 as well as GUI 724 used to present pump-specific information to the operator. The buttons 722 can allow the operator to program the syringe pump 702 for the flow rate, the volume to be infused, and other pump parameters. GUI 724 can present the programmed flow rate, the amount of fluid remaining to be infused, as well as alarms and other information.

The drive head 710 can include a contact plate 726 that has a pushing surface that contacts the outer side 717 of the push-button 709 as the drive head 710 moves forward toward the barrel 703, pushing the plunger 707 into the barrel 703 of the syringe to expel the syringe contents through a fluid administration set tubing 719 to a patient. A drive mechanism 754 is configured to drive the drive head 710 forward. The drive mechanism 754 may include, a drivetrain, for example, gears, axles, shafts, chains, hydraulics, and/or any other components for translating rotational motion of a motor into linear motion of the drive head 710. The drivetrain may include linear actuating components, such as a linear stepper motor, a brushed DC electric motor, a brushless DC electric motor, a servo motor, an AC motor, or any other type of motor.

When the contact plate 726 exerts force against the push-button 709, the force may be detected by force sensor 729 and transmitted to a processor for monitoring. In the case of friction between the stopper 713 and the barrel 703, the force exerted can increase and can be detected by force sensor 729.

Syringe pump 702 is configured with known syringe types containing information such as syringe inner diameter and stroke of the stopper 713. Syringe pump 702 determines the position of stopper 713 based on the movement of drive head 710 based on the syringe type and stored characteristics of the syringe type. Then, syringe pump 702 calculates the volume infused, time elapsed, volume remaining, and time remaining. As the drive head 710 continues to move, a flow rate is determined based on the characteristics of the syringe and the velocity of the drive head 710.

One or more sensors, such as sensor 728, barrel clamp 706, groove 708, claws 712 and 714, and force sensor 729, may measure various characteristics of syringe 701, to obtain a measured value for the characteristic, such as barrel length, barrel outside diameter, flange shape, flange size, plunger shape, plunger size (e.g., diameter), plunger tab thickness, volume per plunger distance moved, driver head height, etc. These sensors may include one or more of an optical sensor, a light source, a pressure sensor, a position sensor (e.g., magnetic linear position sensor indicating the plunger head), force sensor, and the like.

### Example Method for Updating an Operational Parameter of a Remote Medical Device

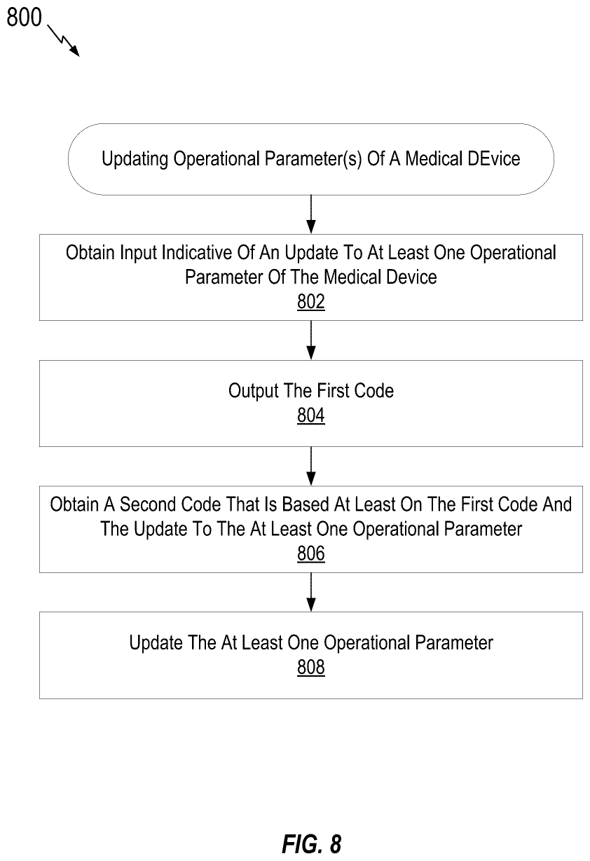

FIG. 8 shows an example method 800 for updating an operational parameter of a remote medical system, for example, infusion system 600 in FIG. 6, or syringe system 702 in FIG. 7.

Method 800 begins at block 802 with obtaining input indicative of an update to at least one operational parameter of a medical device, such as described at step 204 of FIG. 2.

In some aspects, method 800 further comprises generating, at the medical device, the first code, such as described at step 206 of FIG. 2.

Method 800 proceeds at block 804 with outputting a first code, such as described at step 208 of FIG. 2. In some aspects, outputting the first code comprises displaying, on a graphics user interface of the medical device, the first code.

Method 800 then proceeds at block 806 with obtaining a second code that is based at least on the first code and the update to the at least one operational parameter, such as described at step 218 of FIG. 2.

Based on verification of the second code with respect to the first code and the update to the at least one operational parameter, method 800 proceeds at block 808 with updating the at least one operational parameter based on the input, such as described at steps 220-222 of FIG. 2.

In some aspects, verifying the second code comprising: encrypting, using a shared secret key, data comprising the second code to generate a hash digest, such as described at step 342 of FIG. 3C; comparing the hash digest to a pattern requirement, such as described at step 346 of FIG. 3C; and verifying the hash digest based on matching the pattern requirement, such as described at step 348 of FIG. 3C.

In some aspects, the data further comprises the first code. In some aspects, the data further comprises the update. In some aspects, the data further comprises a timestamp associated with the first code and an identifier of the medical device.

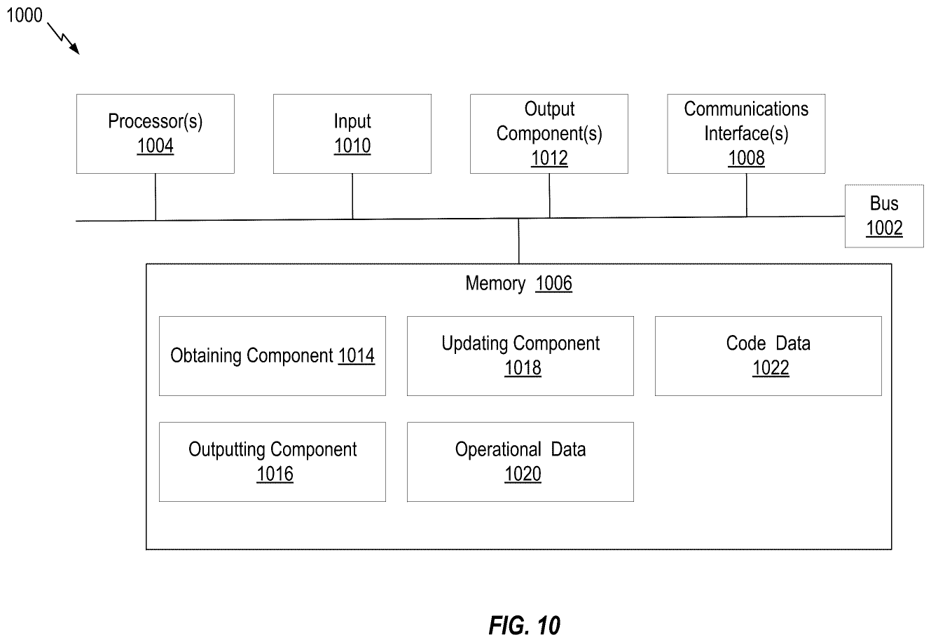

In one aspect, method 800, or any aspect related to it, may be performed by an apparatus, such as computing device 1000 of FIG. 10, which includes various components operable, configured, or adapted to perform the method 800. Computing device 1000 is described below in further detail.

Note that FIG. 8 is just one example of a method, and other methods including fewer, additional, or alternative steps are possible consistent with this disclosure.

### Example Method for Verifying an Update to an Operational Parameter of a Remote Medical Device

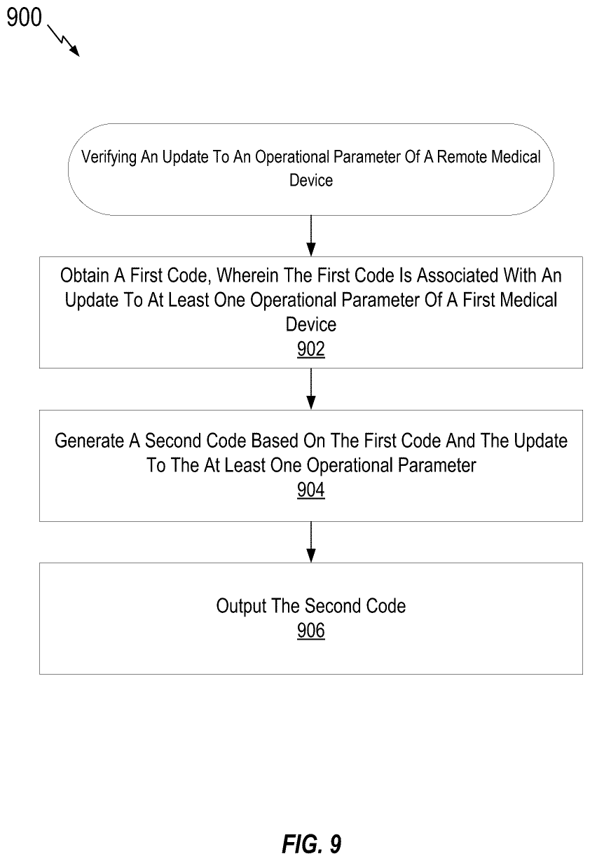

FIG. 9 shows an example method 900 for verification of an update an operational parameter of a remote medical system, such as infusion system 600 in FIG. 6, or syringe system 702 in FIG. 7.

Method 900 begins at block 902 with obtaining a first code, wherein the first code is associated with an update to at least one operational parameter of a first medical device, such as described at step 210 of FIG. 2.

In some aspects, method 900 further comprises verifying the update to the at least one operational parameter of the first medical device, such as described at step 212 of FIG. 2.

In some aspects, verifying the update to the at least one operational parameter of the first medical device comprises: updating, at a virtual medical device associated with a second medical device, the at least one operational parameter, such as described at step 302 of FIG. 3A; comparing, the at least one operational parameter to a device threshold associated with the virtual medical device, such as described at step 303 of FIG. 3A; and verifying the at least one operational parameters based on the at least one operational parameter satisfying the device threshold, such as described at step 306 of FIG. 3A.

Method 900 proceeds with block 904 with generating a second code based at least on the first code and the update to the at least one operational parameter, such as described at step 214 of FIG. 2.

Method 900 then proceeds with block 906 with outputting the second code, such as described at step 216 of FIG. 2. In some aspects, outputting the second code comprises displaying, on a graphical user interface of a second medical device, the second code.

In some aspects, method 900 further comprises authenticating a user of the second medical device, such as described at step 310 of FIG. 3A.

In some aspects, method 900 further comprises signing a message with a digital signature of the authenticated user, wherein the message comprises the at least one operational parameter; and transmitting, the signed message to an electronic medical record associated with a patient associated with the first medical device.

In some aspects, generating the second code comprises: encrypting, using a shared secret key, data comprising the second code to generate a hash digest, such as described at step 322 of FIG. 3B; comparing the hash digest to a pattern requirement, such as described at step 326 of FIG. 3B; and outputting the second code based on the hash digest based on matching the pattern requirement.

In some aspects, the data further comprises the first code. In some aspects, the data further comprises the at least one operational parameter. In some aspects, the data further comprises a timestamp associated with the first code and an identifier of the first medical device.

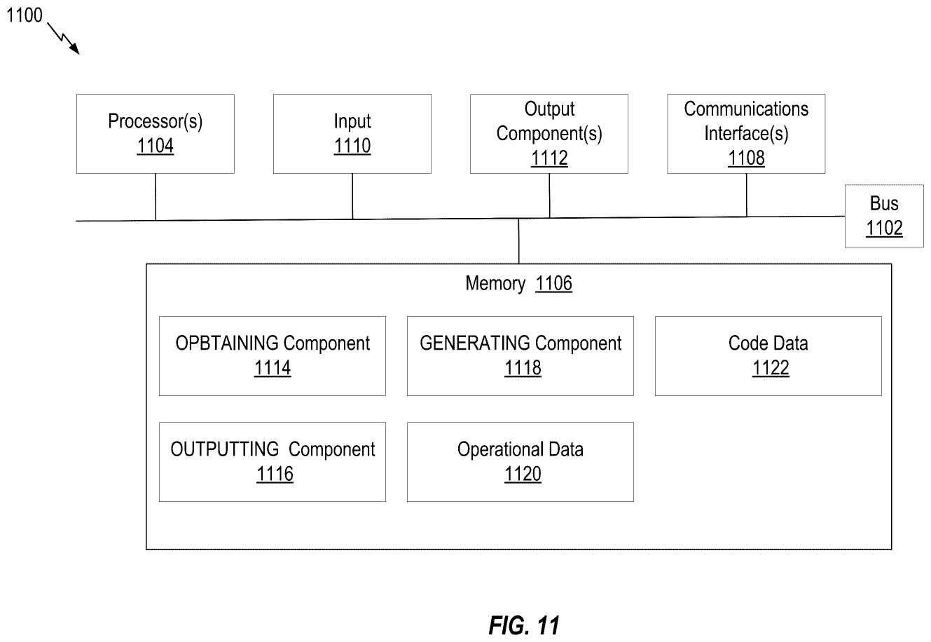

In one aspect, method 900, or any aspect related to it, may be performed by an apparatus, such as computing device 1100 of FIG. 11, which includes various components operable, configured, or adapted to perform the method 900. Computing device 1100 is described below in further detail.

Note that FIG. 9 is just one example of a method, and other methods including fewer, additional, or alternative steps are possible consistent with this disclosure.

### Example Computing Device for Verification of a Remote Medical Device

FIG. 10 depicts an example computing device 1000, such as a medical device, which implements various features and processes described herein, such as infusion system 600 in FIG. 6 and/or syringe pump 702. For example, the computing device 1000 may perform one or more steps of any of workflow 200, or 300, or method 800. The computing device 1000 may include one or more processors 1004, one or more memories 1006, one or more input components 1010, one or more output components 1012, and one or more communication interfaces 1008. Each of these components may be coupled by a bus 1002.

Computing device 1000 may perform these processes based on one or more processors 1004 executing software instructions stored by a computer-readable medium, such as one or more memories 1006. In certain embodiments, one or more processors 1004 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 1008. When executed, software instructions stored in one or more memories may cause one or more processors 1004 to perform one or more processes described herein.

A memory 1006 may include data storage or one or more data structures (e.g., a database, etc.). Computing device 1000 may be capable of receiving information from, storing information in, communicating information to, or searching information stored in the data storage or one or more data structures in one or more memories 1006.

A memory 1006 may include random access memory (RAM), read only memory (ROM), and/or other types of dynamic or static storage devices (e.g., flash memory, magnetic memory, optical memory, etc.), that stores information and/or instructions for use by one or more processors 1004. For example, a memory 1006 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 1006 may include an obtaining component 1014 is configured for obtaining input indicative of an update to at least one operational parameter of a medical device, such as described at step 204 of FIG. 2 or block 802 of FIG. 8, and/or obtaining a second code that is based at least on the first code and the update to the at least one operational parameter, such as described at step 218 of FIG. 2 or block 806 of FIG. 8.

In some aspects, one or more memories 1006 may include an outputting component 1016 is configured for outputting a first code, such as described at step 208 of FIG. 2 or block 804 of FIG. 8.

In some aspects, one or more memories 1006 may include an updating component 1018 is configured for updating the at least one operational parameter based on the input based on verification of the second code with respect to the first code and the update to the at least one operational parameter, such as described at steps 220-222 of FIG. 2 and/or block 808 of FIG. 8.

In some aspects, one or more memories 1006 may include operational data 1020 used by obtaining component 1014 and/or updating component 1018.

In some aspects, one or more memories 1006 may include code data 1022 used by outputting component 1016, and/or a verification component.

One or more processors 1004 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 1010 may include a component that permits computing device 1000 to receive information, such as via user input (e.g., a touch-screen display, a keyboard, a keypad, a mouse, a button, a switch, a microphone, etc.). Further, one or more input components 1010 may include a sensor for sensing information (e.g., a global positioning system (GPS) component, an accelerometer, a gyroscope, an actuator, etc.).

One or more output components 1012 may include a component that provides output information from computing device 1000 (e.g., a display, a speaker, one or more light-emitting diodes (LEDs), etc.).

Communication interface 1008 may include a transceiver-like component (e.g., a transceiver, a separate receiver and transmitter, etc.) that enables computing device 1000 to communicate with other devices, such as via a wired connection, a wireless connection, or a combination of wired and wireless connections. Communication interface 1008 may permit computing device 1000 to receive information from another device and/or provide information to another device. For example, communication interface 1008 may include an Ethernet interface, an optical interface, a coaxial interface, an infrared interface, a radio frequency (RF) interface, a universal serial bus (USB) interface, a Wi-Fi interface, a cellular network interface, and/or the like.

FIG. 11 depicts another example computing device 1100, such as a medical device, which implements various features and processes described herein. For example, the computing device 1100 may perform one or more steps of any of workflow 200 or 300 or method 900. The computing device 1100 may include one or more processors 1104, one or more memories 1106, one or more input components 1110, one or more output components 1112, and one or more communication interfaces 1108. Each of these components may be coupled by a bus 1102.

Computing device 1100 may perform these processes based on one or more processors 1104 executing software instructions stored by a computer-readable medium, such as one or more memories 1106. In certain embodiments, one or more processors 1104 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 1108. When executed, software instructions stored in one or more memories may cause one or more processors 1104 to perform one or more processes described herein.

A memory 1106 may include data storage or one or more data structures (e.g., a database, etc.). Computing device 1100 may be capable of receiving information from, storing information in, communicating information to, or searching information stored in the data storage or one or more data structures in one or more memories 1106.

A memory 1106 may include random access memory (RAM), read only memory (ROM), and/or other types of dynamic or static storage devices (e.g., flash memory, magnetic memory, optical memory, etc.), that stores information and/or instructions for use by one or more processors 1104. For example, a memory 1106 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 1106 may include an obtaining component 1114 is configured for obtaining a first code, wherein the first code is associated with an update to at least one operational parameter of a first medical device, such as described at step 210 of FIG. 2 and/or block 902 of FIG. 9.

In some aspects, one or more memories 1106 may include an outputting component 1116 is configured for outputting the second code, such as described at step 216 of FIG. 2 and/or block 906 of FIG. 9.

In some aspects, one or more memories 1106 may include a generating component 1118 is configured for generating a second code based at least on the first code and the update to the at least one operational parameter, such as described at step 214 of FIG. 2 and/or block 904 of FIG. 9.

In some aspects, one or more memories 1106 may include operational data 1120 used by generating component 1118 and/or a verification component.

In some aspects, one or more memories 1106 may include code data 1122 used by obtaining component 1114, and/or outputting component 1116, and/or generation component 1118.

One or more processors 1104 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 1110 may include a component that permits computing device 1100 to receive information, such as via user input (e.g., a touch-screen display, a keyboard, a keypad, a mouse, a button, a switch, a microphone, etc.). Further, one or more input components 1110 may include a sensor for sensing information (e.g., a global positioning system (GPS) component, an accelerometer, a gyroscope, an actuator, etc.).

One or more output components 1112 may include a component that provides output information from computing device 1100 (e.g., a display, a speaker, one or more light-emitting diodes (LEDs), etc.).

Communication interface 1108 may include a transceiver-like component (e.g., a transceiver, a separate receiver and transmitter, etc.) that enables computing device 1100 to communicate with other devices, such as via a wired connection, a wireless connection, or a combination of wired and wireless connections. Communication interface 1108 may permit computing device 1100 to receive information from another device and/or provide information to another device. For example, communication interface 1108 may include an Ethernet interface, an optical interface, a coaxial interface, an infrared interface, a radio frequency (RF) interface, a universal serial bus (USB) interface, a Wi-Fi interface, a cellular network interface, and/or the like.

### EXAMPLE CLAUSES

Implementation examples are described in the following numbered clauses:

Clause 1: A method, comprising: obtaining input indicative of an update to at least one operational parameter of a medical device; outputting a first code; obtaining a second code that is based at least on the first code and the update to the at least one operational parameter; and based on verification of the second code with respect to the first code and the update to the at least one operational parameter, updating the at least one operational parameter based on the input.

Clause 2: The method of clause 1, wherein outputting the first code comprises displaying, on a graphical user interface of the medical device, the first code.

Clause 3: The method of any one of clauses 1-2, further comprising generating, at the medical device, the first code.

Clause 4: The method of any one of clauses 1-3, further comprising verifying the second code comprising: encrypting, using a shared secret key, data comprising the second code to generate a hash digest; comparing the hash digest to a pattern requirement; and verifying the hash digest based on matching the pattern requirement.

Clause 5: The method of clause 4, wherein the data further comprises the first code.

Clause 6: The method of any one of clauses 4-5, wherein the data further comprises the update.

Clause 7: The method of clause 6, wherein the data further comprises a timestamp associated with the first code and an identifier of the medical device.

Clause 8: A method, comprising: obtaining a first code, wherein the first code is associated with an update to at least one operational parameter of a first medical device; generating a second code based at least on the first code and the update to the at least one operational parameter; and outputting the second code.

Clause 9: The method of clause 8, wherein outputting the second code comprises displaying, on a graphical user interface of a second medical device, the second code.

Clause 10: The method of any one of clauses 8-9, further comprising verifying the update to the at least one operational parameter of the first medical device.

Clause 11: The method of clause 10, wherein verifying the update to the at least one operational parameter of the first medical device comprises: updating, at a virtual medical device associated with a second medical device, the at least one operational parameter; comparing, the at least one operational parameter to a device threshold associated with the virtual medical device; and verifying the at least one operational parameters based on the at least one operational parameter satisfying the device threshold.

Clause 12: The method of clause 11, further comprising authenticating a user of the second medical device.

Clause 13: The method of clause 12, further comprising: signing a message with a digital signature of the authenticated user, wherein the message comprises the at least one operational parameter; and transmitting, the signed message to an electronic medical record associated with a patient associated with the first medical device.

Clause 14: The method of any one of clauses 8-13, wherein generating the second code comprises: encrypting, using a shared secret key, data comprising the second code to generate a hash digest; comparing the hash digest to a pattern requirement; and outputting the second code based on the hash digest based on matching the pattern requirement.

Clause 15: The method of clause 14, wherein the data further comprises the first code.

Clause 16: The method of any one of clauses 14-15, wherein the data further comprises the at least one operational parameter.

Clause 17: The method of clause 16, wherein the data further comprises a timestamp associated with the first code and an identifier of the first medical device.

Clause 18: An infusion system, comprising: an infusion pump configured to deliver fluid to a patient; and an infusion pump controller configured to perform a method in accordance with any one of Clauses 1-17.

Clause 19: A medical device, comprising: a processing system that includes one or more processors and one or more memories coupled with the one or more processors, the processing system configured to cause the medical device to perform a method in accordance with any one of Clauses 1-17.

Clause 20: A processing system, comprising: memory comprising computer-executable instructions; and one or more processors configured to execute the computer-executable instructions and cause the processing system to perform a method in accordance with any one of Clauses 1-17.

Clause 21: A processing system, comprising means for performing a method in accordance with any one of Clauses 1-17.

Clause 22: A non-transitory computer-readable medium storing program code for causing a processing system to perform the steps of any one of Clauses 1-17.

Clause 23: A computer program product embodied on a computer-readable storage medium comprising code for performing a method in accordance with any one of Clauses 1-17.

### ADDITIONAL CONSIDERATIONS

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database, or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A medical device comprising a processing system that includes one or more processors and one or more memories coupled with the one or more processors, the processing system configured to cause the medical device to:
obtain input indicative of an update to at least one operational parameter of a medical device;
output a first code;
obtain a second code that is based at least on the first code and the update to the at least one operational parameter; and
based on verification of the second code with respect to the first code and the update to the at least one operational parameter, update the at least one operational parameter based on the input.

2. The medical device of claim 1, wherein to output the first code, the processing system is configured to cause the medical device to display, on a graphical user interface of the medical device, the first code.

3. The medical device of claim 1, wherein the processing system is configured to cause the medical device to generate, at the medical device, the first code.

4. The medical device of claim 1, the processing system is further configured to cause the medical device to verify the second code comprising:
encrypt, using a shared secret key, data comprising the second code to generate a hash digest;
compare the hash digest to a pattern requirement; and
verify the hash digest based on matching the pattern requirement.

5. The medical device of claim 4, wherein the data further comprises the first code.

6. The medical device of claim 4, wherein the data further comprises the update.

7. The medical device of claim 6, wherein the data further comprises a timestamp associated with the first code and an identifier of the medical device.

8. A first medical device comprising a processing system that includes one or more processors and one or more memories coupled with the one or more processors, the processing system configured to cause the first medical device to:
obtain a first code, wherein the first code is associated with an update to at least one operational parameter of a second medical device,
generate a second code based at least on the first code and the update to the at least one operational parameter; and
output the second code.

9. The first medical device of claim 8, wherein to output the second code, the processing system is configured to cause the first medical device to display, on a graphical user interface of the first medical device, the second code.

10. The first medical device of claim 8, wherein the processing system is configured to cause the first medical device to verify the update to the at least one operational parameter of the second medical device.

11. The first medical device of claim 10, wherein to verify the update to the at least one operational parameter of the second medical device, the processing system is configured to cause the first medical device to:
update, at a virtual medical device associated with the second medical device, the at least one operational parameter;
compare, the at least one operational parameter to a device threshold associated with the virtual medical device; and
verify the at least one operational parameter based on the at least one operational parameter satisfying the device threshold.

12. The first medical device of claim 11, wherein the processing system is configured to cause the first medical device to authenticate a user of the first medical device.

13. The first medical device of claim 12, wherein the processing system is configured to cause the first medical device to:
sign a message with a digital signature of the authenticated user, wherein the message comprises the at least one operational parameter; and
transmit, the signed message to an electronic medical record associated with a patient associated with the second medical device.

14. The first medical device of claim 8, wherein to generate the second code, the processing system is configured to cause the first medical device to:
encrypt, using a shared secret key, data comprising the second code to generate a hash digest;
comparing the hash digest to a pattern requirement; and
outputting the second code based on the hash digest based on matching the pattern requirement.

15. The first medical device of claim 14, wherein the data further comprises the first code.

16. The first medical device of claim 14, wherein the data further comprises the at least one operational parameter.

17. The first medical device of claim 16, wherein the data further comprises a timestamp associated with the first code and an identifier of the second medical device.

18. A method, comprising:
obtaining input indicative of an update to at least one operational parameter of a medical device;
outputting a first code;
obtaining a second code that is based at least on the first code and the update to the at least one operational parameter; and
based on verification of the second code with respect to the first code and the update to the at least one operational parameter, updating the at least one operational parameter.

19. The method of claim 18, further comprising generating, at the medical device, the first code.

20. The method of claim 18, further comprising verifying the second code comprising:
encrypting, using a shared secret key, data comprising the second code to generate a hash digest;
comparing the hash digest to a pattern requirement; and
verifying the hash digest based on matching the pattern requirement.
